(19) United States
(12) Patent Application Publication
(10) Pub. No.: US 2016/0296412 A1
(43) Pub. Date: Oct. 13, 2016

(54) FOOT ORTHOTIC DEVICE AND SYSTEM AND METHODS OF MAKING AND USING THEREOF

(71) Applicant: Mia Thomas, Ithaca, NY (US)

(72) Inventor: Mia Thomas, Ithaca, NY (US)

(21) Appl. No.: 15/094,499

(22) Filed: Apr. 8, 2016

Related U.S. Application Data
(60) Provisional application No. 62/144,863, filed on Apr. 8, 2015.

(51) Int. Cl. A61H 7/00; A43B 17/00; A43B 7/14; A61B 5/11; A41B 11/00; A43B 7/16

(52) U.S. Cl. CPC A61H 7/001; A41B 11/007; A43B 7/16; A43B 7/147; A61B 5/112; A43B 17/00; A61H 2205/12

(57) ABSTRACT

A foot orthotic device that can be adhered to and detached from an insole for treating subjects susceptible to toe walking.

### CROSS-REFERENCES TO RELATED APPLICATIONS

This application claims the benefit of U.S. Provisional Patent Application No. 62/144,863, entitled “FOOT ORTHOTIC DEVICE AND SYSTEM AND METHODS OF MAKING AND USING THEREOF”, filed Apr. 8, 2015, which is incorporated herein by reference in its entirety.

### BACKGROUND OF THE INVENTION

The present invention is in the technical field of health. More particularly, the present invention is in the technical field of foot orthotics that provide sensory input and are useful in reducing self-stimulatory behaviors in subjects suffering from a neurological disorder.

Self-stimulatory behaviors, such as toe-walking and finger flicking, have been associated with autism spectrum disorder (ASD), sensory processing disorder (SPD), duplications in the long arm the 1q21.1 chromosome, vestibular system disorders, hyposensitivity, hypersensitivity and central or peripherial nervous system disorders. The Center for Disease Control reports that 1 in every 68 children in the United States have been diagnosed with Autism Spectrum Disorder (ASD). ASD is defined as persistent deficits in social communication and social interaction across multiple contexts accompanied by restricted repetitive patterns of behavior, interests, or activities that are present during the early stages of development. While most repetitive or self-stimulatory behaviors exhibited by participants with autism include activities which may be perceived as harmless (e.g. finger flipping, repetitive vocalizations, clicking, spinning objects, or gazing), some (e.g. toe-walking) can affect the participants' quality of life and functional capabilities. Of children diagnosed with autism, 19% exhibit toe-walking. If this condition continues into adolescence without interventions, it can lead to more serious conditions such as contractures in the triceps surae, early arthritis in the bones of the midfoot, medial tibial stress syndrome, and Achilles tenosynovitis.

Health care professionals are currently offering treatments, which have only proven to give short-term effects. These include: physical therapy focusing on stretching and massage the Gastrocnemius-Salues Complex to prevent contractures or ultrasound and laser therapy to promote healing in Achilles tenosynovitis; surgery to lengthen patients Achilles tendon while restricting any plantar flexion; serial casting; Onabotulinumtoxin A injections to a subject's gastrocnemius and solus muscles; and wearing ankle foot orthotics, and/or night splints for several months. Some of these procedures are followed by constant use of short leg braces. In addition, pharmacotherapy is also used to help reduce self-stimulatory behaviors. However, it is unknown as to whether these directly affect the behavior, decrease the patient's anxiety, or the patient's motor function in general. Common medications prescribed to reduce anxiety and possibly decrease self-stimulatory behaviors are selective serotonin reuptake inhibitors (SSRI), Naltrexone and Clomipramine. These treatments are not only invasive and potentially painful to the patient, but they are also extremely expensive.

Insoles for everyday footwear are well known (see e.g. those described in U.S. Pat. No. 4,823,483) as well as those developed for particular medical conditions (see e.g. U.S. Pat. No. 6,481,120 describing insoles for subjects suffering from arthritis and diabetes). Both types of insoles are usually mass produced, so there is very little customization available for the particular user. In addition, these types of products can be contra-indicated for neurological conditions and symptoms, such as toe-walking. To produce custom made foot orthoses for every type of footwear, or changing foot condition is not practical.

Therefore, there is a need for an inexpensive, potentially removable and resusable series of foot orthotic devices which provide control for self-stimulatory behaviors such as toe walking, in subjects with a neurological disorder. The present invention provides a solution by accommodating different foot types and mechanics by using a series of inexpensive, individual orthotic devices which are of self-customizable by their placement in the heel area of the insole of an article of footwear that correspond to the area in a subject's foot which are most sensitive to stimulation; to provide increased stimulation to the subject with a normal walking gait pattern in order to decrease self-stimulatory behaviors in the subject.

### BRIEF SUMMARY OF THE INVENTION

The present provides an orthotic device comprising: a substrate having 1) an upper, raised proximal surface for displacing the surface of the heel of a subject's foot of a height sufficient for the subject to feel pressure from the device, and 2) a distal surface for contact with an insole of an article of footwear. The present invention also provides a foot orthotic system comprising at least one device of the present invention and an insole of an article of footwear. The present invention also provides methods for treating or preventing self-stimulating behavior in a subject with autism spectrum disorder (ASD), sensory processing disorder (SPD), having duplications in the long arm the 1q21.1 chromosome, vestibular system disorders, hyposensitivity, hypersensitivity or central or peripherial nervous system disorders comprising using a system of the present invention. The present invention also provides a method for making a system of the present invention.

### BRIEF DESCRIPTION OF THE DRAWINGS

FIG. 1a is a top view of a first embodiment of an orthotic device of the present invention;

FIG. 1b is a side bottom perspective view thereof;

FIG. 2 is a top-front-left side perspective view of a first embodiment of a left insole system using the devices of the present invention;

FIG. 3 is a top-front-left side perspective view of a second embodiment of a left insole system using the devices of the present invention;

FIG. 4 is a top-front-left side perspective view of a third embodiment of a left insole system using the devices of the present invention;

FIG. 5 is a top view of a first embodiment of an orthotic device of the present invention;

FIG. 6 is a side bottom perspective view thereof;

FIG. 7 is a side bottom perspective view thereof wherein the bottom surface is not shown;

FIG. 8 is a top-front-left side perspective view of a first embodiment of a generic insole system using the devices of the present invention;

FIG. 9 is a top-front-left side perspective view of a second embodiment of a generic insole system using the devices of the present invention; and

FIG. 10 is a top-front-left side perspective view of a second embodiment of a generic insole system using the devices of the present invention.

### DEFINITIONS

The term “self-stimulating behavior” or “stereotypy” refers to repetitive body movements or repetitive movement of objects. This behavior is common in many individuals with developmental disabilities and neurological disorders, including individuals diagnosed with autism.

### DETAILED DESCRIPTION OF THE INVENTION

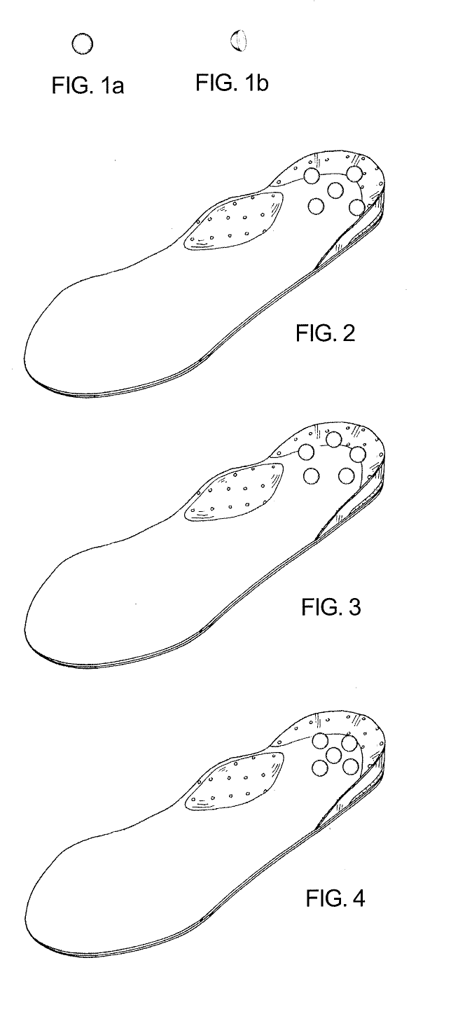

The invention will now be described in more detail with reference to the drawings. FIG. 1a is a top view and FIG. 1b is a side view of a hemispherical embodiment of a orthotic device of the present invention. The orthotic device is comprised of a substrate with an upper, raised proximal surface for displacing the surface of the heel of a subject's foot. The shape and size of the device is sufficient for the subject to feel pressure from the device during a normal walking gait. The device also has a distal surface for contact with an insole of an article of footwear. In further detail to FIG. 1, the shape and size (height, diameter and distal surface area) of the device can be adjusted to the size of the subject's foot. Thus, for adult subjects, the diameter of the device may range from about 0.1 of an inch, to about 0.15 of an inch, to about 0.25 of an inch, to about 0.3 of an inch, to about 0.35 of an inch, to about 0.4 of an inch, to about 0.45 of an inch, to about 0.5 of an inch, to about 0.55 of an inch, to about 0.6 of an inch, to about 0.65 of an inch, to about 0.7 of an inch, to about 0.75 of an inch, to about 0.8 of an inch, to about 0.85 of an inch, to about 0.9 of an inch, to about 0.95 of an inch, to about 1 inch. For adolescent subjects, the diameter of the device may range from about from about 0.1 of an inch, to about 0.15 of an inch, to about 0.25 of an inch, to about 0.3 of an inch, to about 0.35 of an inch, to about 0.4 of an inch, to about 0.45 of an inch, to about 0.5 of an inch. In some embodiments, distal surface area of the device attached to the insole will be less than the area of the heel of the subject's foot. In some embodiments, the distal surface area of the device will be less or equal to half, a third, a quarter, a fifth, a sixth, a seventh, an eighth, a ninth or a tenth of the area of the subject's heel. In some embodiments, the distal surface area of the devices ranges from about 0.01 in.², to about 0.02 in.², to about 0.03 in.², to about 0.04 in.², to about 0.05 in.², to about 0.06 in.², to about 0.07 in.², to about 0.08 in.² to about 0.09 in.², to about 0.1 in.², to about 0.11 in.², to about 0.12 in.², to about 0.13 in.², to about 0.14 in.², to about 0.15 in.², to about 0.16 in.² to about 0.17 in. ², to about 0.18 in.², to about 0.19 in.², to about 0.20 in.², to about 0.21 in.², to about 0.22 in.², to about 0.23 in.², to about 0.24 in.² to about 0.25 in.², to about 0.26 in.², to about 0.27 in.², to about 0.28 in.², to about 0.29 in.², to about 0.30 in.², to about 0.31 in.², to about 0.32 in.² to about 0.33 in.², to about 0.34 in.², to about 0.35 in.², to about 0.36 in.², to about 0.37 in.², to about 0.38 in.², to about 0.39 in.², to about 0.40 in.² to about 0.41 in.², to about 0.42 in.², to about 0.43 in.², to about 0.44 in.², to about 0.45 in.², to about 0.46 in.², to about 0.47 in.², to about 0.48 in.² to about 0.49 in.², to about 0.5 in.², from about 0.51 in.², to about 0.52 in.², to about 0.53 in.², to about 0.54 in.², to about 0.55 in.², to about 0.56 in.², to about 0.57 in.², to about 0.58 in.² to about 0.59 in.², to about 0.6 in.², to about 0.61 in.², to about 0.62 in.², to about 0.63 in.², to about 0.64 in.², to about 0.65 in.², to about 0.66 in.² to about 0.67 in.², to about 0.68 in.², to about 0.69 in.², to about 0.70 in.², to about 0.71 in.², to about 0.72 in.², to about 0.73 in.², to about 0.74 in.² to about 0.75 in.², to about 0.76 in.², to about 0.77 in.², to about 0.78 in.², to about 0.79 in.², to about 0.80 in.², to about 0.81 in.², to about 0.82 in.² to about 0.83 in.², to about 0.384 in.², to about 0.85 in.², to about 0.86 in.², to about 0.87 in.², to about 0.88 in.², to about 0.89 in.², to about 0.90 in.² to about 0.91 in.², to about 0.92 in.², to about 0.93 in.², to about 0.94 in.², to about 0.95 in.², to about 0.96 in.², to about 0.97 in.², to about 0.98 in.² to about 0.99 in.², to about 1 in.².

In some of embodiments, the height of the device is sufficient for the subject to feel pressure during a normal walking gait. In some embodiments, the height of the device (at its highest point, which may vary with the shape) may range from about 0.1 of an inch, to about 0.15 of an inch, to about 0.25 of an inch, to about 0.3 of an inch, to about 0.35 of an inch, to about 0.4 of an inch, to about 0.45 of an inch, to about 0.5 of an inch. For adolescent subjects, the height may range from about 0.1 of an inch, to about 0.15 of an inch, to about 0.2 of an inch, to about 0.25 of an inch.

In addition, it will be appreciated by persons knowledgeable in the art, that the shape of device component is not limited, but may be configured to any shape which may be attachable to an insole of an article of footwear. Examples of such shapes include, but are not limited to a hemisphere, a cylinder, a pyramid, a cone and a cube. In some embodiments, the shape of the device may have hard edges. In some embodiments, the shape of the device may have soft edges.

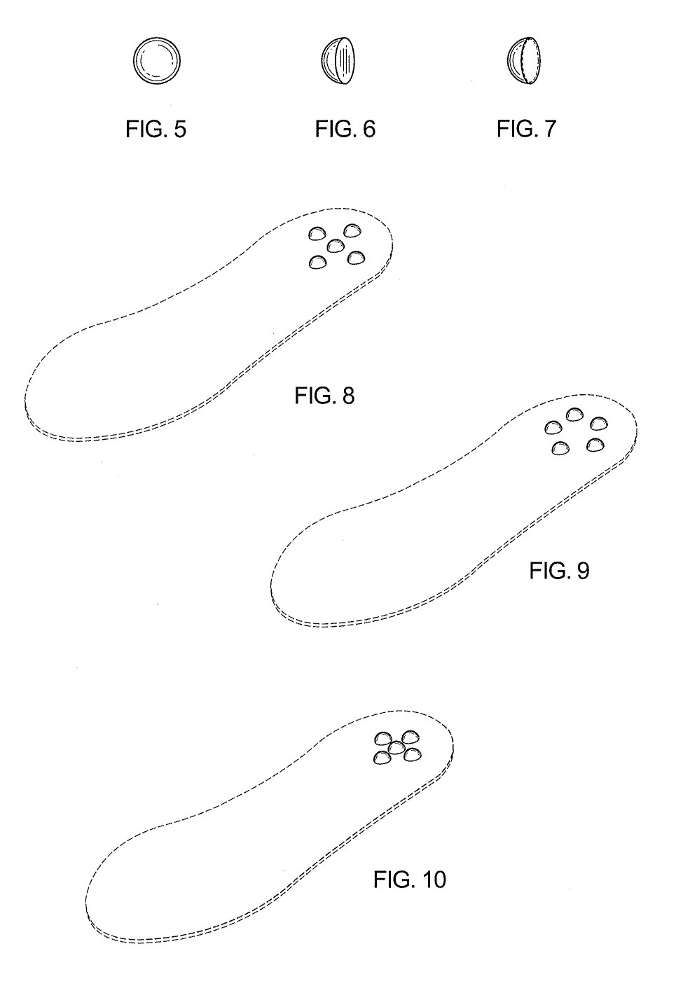

The device of FIGS. 1a, 1b, and 5-7 is made of a semi-rigid material, substantially non-compressible material hard enough to support the weight of an active, full grown adult but at the same time flexible enough to allow the foot to work naturally and comfortably when it is in contact it and the insole of an article of footwear. For example, in some embodiments, the material exhibits less than about 1%, to about 2%, to about 3%, to about 4%, to about 5%, to about 6%, to about 7%, to about 8%, to about 9%, to about 10%, to about 11%, to about 12%, to about 13%, to about 14%, or to about 15% compression when subjected to a tensile load of about 250 pounds. Examples of such materials, include but are not limited to a plastic, glass, aramid, rubber, metal, and wood that would allow patients of varying weight to feel the pressure placed by the device. One of skill in the art would recognize that by varying the value of the 1) hardness, 2) thickness, or 3) diameter of the device, or its shape, the relative level of pressure on the corresponding portion of the heel can be increased or decreased accordingly.

In accordance with the present invention, at least one of the devices are placed in an insole in the area of the heel to form an orthotic system of the present invention. FIGS. 3-4 and 8-10 are perspective views of heel patterns for placement of the orthotic devices. Accordingly, in some embodiments, the device has an adhesive means layer on the distal surface which can adhere the device to an insole of an article of footwear and reduce its movement within said article of footwear. Depending on the adhesive used, the device may be removable, reattachable and/or reusable. Adhesive means are known in the art and examples of adhesive means, include but are not limited to a chemical adhesive glue, Velcro™, magnets, metal fasteners or any other fastening system. In some embodiments, the adhesive means may comprise at least one protective cover removeably disposed over said adhesive layer which, when removed, exposes said adhesive layer.

Referring now to FIG. 2, there is shown a diagram of a typical left insole for an article of footwear has a heel portion that has five devices (as individually shown in FIGS. 1 and 5-7) placed in a pattern in the heel area. In other groups of embodiments, the system has one, two, three, four, six, seven, eight, nine or ten devices placed in the heel of an insole. In some embodiments, the devices can be of different heights, widths, shapes or materials.

In alternative embodiments in FIGS. 3, 4 and 8-10, the devices can be placed in alterative patterns in the heel area of an insole. In certain embodiments, the devices are placed in the area of the heel that receive the most stimulation during normal walking. In other groups of embodiments, the devices may be placed in areas of the heel that are most sensitive to stimulation to the subject during a normal walking gait.

The construction details of the inventions shown in FIGS. 3, 4, and 8-10 may be the same or different from those shown in FIG. 2, as well as for a right insole. The pattern for one foot need not be the same as the other foot. The pattern of placement of the devices may be symmetric or asymmetric.

The advantages of the present invention include, without limitation, improving gait patterns in patients with neurological disorders with the addition of a heel strike phase. The addition of this phase will increase their dorsiflexion during gait, which will decrease the hip flexion during the swing phase, and increase their knee extension upon ground contact to normal ranges of motion—all of these adjustments will contribute in increasing their stride length.

These orthotics will improve the quality of life for the autistic patients who experience the psychological and physiological pains that come with self-stimulatory behaviors, and specifically toe-walking. The shift to a more normal gait pattern will decrease the amount of potential injuries, including anterior tibial stress syndrome, stress fractures to the tibia, joint pains, muscle fatigue, as well as reduce the stigmas placed on them when toe-walking.

### EXAMPLE

The following example illustrates the treatment to reduce toe-walking as a self-stimulatory behavior. Rubber hemispherical devices as shown in FIGS. 1 and 5-7, were adhered to the heel portion of an insole of a shoe in the three patterns shown in FIGS. 2-4 and 8-10. The three patterns correspond to areas of the heel that receive the most pressure during a typical walking gait. The patterns can be varied as patients with ASD can satiate on a single stimulus. The patterns give the subject increased tactile stimulation by targeting Merkel's disc (SA I) during the swing phase of gait and Ruffini endings (SA II) mechanoreceptors in the stance phase.

The inclusion criteria for subjects was a person who had been diagnosed with autism, exhibited toe-walking, and had full ankle range of motion. Exclusion criteria included: not being able to perform active ankle dorsiflexion and not being able to achieve full ankle range of motion.

The subject was an 8 year-old boy who is generally non-verbal, but able to speak simple phrases (e.g. yes, no, all done). The subject exhibited a moderate amount of toe-walking that was mostly associated with excitement. The study utilized basic video recordings of the research sessions to analyze gait and count the number of heel strikes versus heel touches in 143 steps.

The study used an ABA design with one week after the first baseline measurement to wean the participant onto the treatment and one week after the treatment measurements to decrease the risk of carry-over of the treatment. All sessions were performed on Monday, Wednesday, and Friday evening at 5:30 pm at the subject's house. Every session included a 10-minute warm up walk in the hallway followed by 7 minutes of walking on the treadmill. The participant was videotaped for 143 steps while on the treadmill; these videos were then analyzed by two researchers, both of which were present at the data collection.

Mean values were calculated for the number of forefoot and hind foot strikes for each session as well as each week. The mean difference was calculated to determine any changes in the number of hind foot versus forefoot strikes between treatment and baseline measurements.

Inter-rater and intra-rater reliability analyses were performed to determine the degree of concordance between researchers and within sessions when defining hind foot versus forefoot strikes in the video analysis.

The ICC values calculated showed that there were no correlations between the researchers measurements; therefore, the objective measurements presented are not significant. There was a 56% decrease in the mean number of toe-strikes between the initial baseline assessment and the treatment week. When compared to the final baseline measurements, the treatment week showed a 24% decrease in the number of toe-strikes.

During the initial assessment, the participant displayed an irregular gait pattern with both of his lower extremities externally rotated for a majority of the session, with more rotation noted on the right. Because of this external rotation the participant would often strike with the lateral aspect of his mid or hind foot on the right, while on the left the participant would try to correct the rotation before and after making initial contact. When the participant did exhibit what resembled a typical gait, he continued to have decreased dorsiflexion during the swing phase and upon heel strike. Generally, the left lower extremity exhibited a greater number of heel strikes with increased dorsiflexion present upon initial contact. The inconsistencies seen between the baseline measurements may be due to the participant's irregular gait pattern and inconsistent toe-walking behaviors; or it may be due to a carry-over effect from the use of the treatment.

Thus, the present invention can help improve quality of life, prevent invasive surgeries, and reduce the risk of early arthritis or stress fractures in patients with autism who exhibit toe-walking and other self-stimulatory behaviors seen in patients with autism (i.e. rocking, finger flapping) to provide them with the stimulation they desire in a less distracting manner so they can participate in less restrictive environments.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. Further, it is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An orthotic device comprising: a substrate having 1) an upper, raised proximal surface for displacing the surface of the heel of a subject's foot of a shape and size sufficient for the subject to feel pressure from the device during a normal walking gait, and 2) a distal surface for contact with an insole of an article of footwear.

2. The device of claim 1, wherein said pressure is sufficient to prevent or treat toe-walking.

3. The device of claim 1, wherein said height at its highest point is less than or equal to about 0.25 inches.

4. The device of claim 1, wherein said distal surface area is less than or equal to about 0.5 inches².

5. The device as claimed in claim 1, having a shape selected from the group consisting of a hemisphere, a cylinder, a pyramid, a cone and a cube.

6. The device of claim 1, wherein the substrate is made of a semi-rigid, substantially compression resistant material.

7. The device of claim 1, wherein said material exhibits less than 15% compression when subjected to a tensile load of about 250 pounds.

8. The device of claim 1, wherein said rigid, substantially compression resistant material is selected from the group consisting of plastic, glass, aramid, rubber, metal, and wood.

9. The device of claim 1, optionally comprising an adhesive means layer on the distal surface thereof, and optionally at least one protective layer removably disposed over said adhesive layer, which, when removed, exposes said adhesive layer.

10. The device of claim 1, wherein said adhesive is sufficient to adhere said device to an insole of an article of footwear or reduce its movement within said article of footwear.

11. The device of claim 1, wherein said adhesive layer means allows for the device to be removably insertable into an article of footwear.

12. A foot orthotic system comprising at least one device of claim 1 and an insole of an article of footwear.

13. The system of claim 12, comprising a plurality of devices of claim 1.

14. The system of claim 12, made by adhering a device of claim 1 to the insole of an article of footwear.

15. The system of claim 12, wherein said device is disposed under the heel of the foot.

16. The system according to claim 12, wherein said device is adhered to the insole under Merkel's disc and Ruffini ending receptors on the subject's foot.

17. The system of claim 12 wherein said article of footwear is a sock or a shoe.

18. A method for treating self-stimulating behavior in a subject with autism spectrum disorder (ASD), sensory processing disorder (SPD), having duplications in the long arm the 1q21.1 chromosome, vestibular system disorders, hyposensitivity, hypersensitivity or central or peripherial nervous system disorders comprising using a system of any one of claims 12-18.

19. The method of claim 18, wherein the self-stimulatory behavior is toe-walking.

20. A method for making a system of claim 13, comprising adhering a device of claim 1 to the insole of an article of footwear.
